(19) United States
(12) Patent Application Publication
(10) Pub. No.: US 2024/0052003 A1
(43) Pub. Date: Feb. 15, 2024

(54) COMPOSITIONS COMPRISING PEDF-DERIVED SHORT PEPTIDES (PDSP) AND USES THEREOF

(71) Applicant: BRIM Biotechnology, Inc., Taipei (TW)

(72) Inventors: Frank Wen-Chi Lee, Bedford, MA (US); Wayne W.C. Liaw, San Diego, CA (US); Ping-Yen Huang, Taipei (TW); Hsiao-Han Wang, Taipei (TW)

(73) Assignee: BRIM Biotechnology, Inc., Taipei (TW)

(21) Appl. No.: 17/766,250

(22) PCT Filed: Dec. 4, 2020
(86) PCT No.: PCT/US2020/063182

§ 371 Date: Apr. 3, 2022

Related U.S. Application Data
(60) Provisional application No. 62/911,367, filed on Oct. 6, 2019.

(51) Int. Cl. C07K 14/435; A61K 31/455; A61K 31/4172; A61K 47/10

(52) U.S. Cl. CPC C07K 14/435; A61K 31/455; A61K 31/4172; A61K 47/10

(57) ABSTRACT

An aqueous formulation includes a PEDF-derived short peptide (PDSP) having the sequence of one of SEQ ID NO: 1, 2, 3, 5, 6, 8, or 9; histidine having a concentration of 1 mM-100 mM; and an antioxidant and optionally a non-ionic tonicity agent. The pH value is around 5-9. The antioxidant is nicotinamide, which is at a concentration of 50 mM-1000 mM. The non-ionic tonicity agent is sorbitol, which is at a concentration of 0 mM-500 mM. A concentration of the PDSP is 0.01%-1% w/v.

### BACKGROUND OF INVENTION

### Field of Invention

This invention relates to compositions of PEDF-derived short peptides, particularly to formulations of such peptides and uses thereof.

### Background

Human Pigment Epithelium-derived Factor (PEDF) is a secreted protein of 418 amino acids, with a molecular weight of about 50 kDa. PEDF is a multifuctional protein with many biological functions (see U.S. Patent Application Publication No. 2010/0047212). Different peptide regions of the human PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotropic properties.

Human PEDF-derived short peptides (PDSPs) have been found to be promising therapeutics for treating or preventing various diseases or disorders. For example, PDSPs are found to be effective in promoting muscle regeneration or arteriogenesis (U.S. Pat. No. 9,884,012), treating alopecia and/or hair depigmentation (U.S. Pat. No. 9,938,328), treating osteoarthritis (U.S. Pat. No. 9,777,048), preventing or ameliorating skin aging (U.S. Pat. No. 9,815,878), treating liver cirrhosis (U.S. Pat. No. 8,307,446), or treating various eye diseases or conditions (e.g., retinal degeneration, Meibomian glad disease, dry eye). Corresponding mouse PEDF-derived short peptides (moPDSPs) arer also found to have the same therapeutic effects. However, preparations of these peptide were found to lack long-term stabilities. Therefore, there is a need for better formulations for this promising biopharmaceutical product.

### SUMMARY OF THE INVENTION

Embodiments of the invention relate to formulations for a PEDF-derived short peptide (PDSP), including SEQ ID NO: 1 (39-mw), SEQ ID NO: 2(34-mw), SEQ ID NO: 3 (29-mer), SEQ ID NO: 5 (24-mer), SEQ ID NO: 6 (20-mer), SEQ ID NO: 8 (mo29-mer), and SEQ ID NO: 9 (mo20-mer), wherein mo29-mer and mo20-mer are the mouse PDSPs corresponding to the hum 29-mer and 20-mer, respectively.

One aspect of the invention relate to a aqueous formulation that includes a PDSP having the sequence of one of SEQ ID NO: 1, 2, 3, 5, 6, 8, or 9; histidine having a concentration of 1 mM-100 mM and a antioxidant and optionally a non-ionic tonicity agent. The antioxidant is ascorbic acid or nicotinamide. The non-ionic tonicity agent is sorbitol, dextrose, glycerin, manitol, potassium chloride, sodium chloride, ethylene glycol, or propylene glycol.

According to some embodiments of the invention, the pH value of the aqueous formulations may be around 5-9, preferably around 6.5-7.5. The non-ionic tonicity agent is sorbitol, which is at a concentration of 0 mM-500 mM. The antioxidant is nicotinamide, which is at a concentration of 50 mM-1000 mM. A concentration of the PDSP may be 0.01%-1% w/v.

Other aspects of the invention would be apparent from the following description and the accompanying drawings.

### BRIEF DESCRIPTION OF THE DRAWINGS

FIG. 1 shows a schematic illustrating a testing protocol for assessing the stabilities of various formulations of PDSP solutions. Different PDSP solutions were prepared according to the study design. The pH values of PDSP solutions were adjusted with 1N HCl or 2N NaOH, filtered through a 0.2 μm syringe filter, and placed in a 50 ml glass bottle. The filtered PDSP solutions were stirred at 1,150 RPM at room temperature. Aliquots of 400 μl PDSP solutions were collected at different time points (every half a hour until 7 or 9 hours) ad centrifuged at 1,3000 rpm to observe whether my precipitation had appeared. The stirring of PDSP solutions was continued, and the precipitation was investigated at 10-, 12-, 13- and 24-hour time points. The times for the appearance of suspended matter, precipitation, and turbidity were recorded.

FIG. 2 shows results from stability tests of PDSP formulations prepared in 10 mM Citrate buffer with 0.85% NaCl, pH6.0 and in 20 mM Histidine buffer with different concentrations of Nicotinamide, pH7.0, under continuously stirring conditions. PDSP prepared in these different formulations were each placed in a 50 mL beaker after filtration, and then the solutions were stirred at 1,150 RPM at room temperature. These solutions were investigated every half a hour for the first 7 hours, and the continuous observation was proceeded after 12-hour after the start of the stirring.

FIG. 3 shows results of stability tests of PDSP formulations prepared with different concentrations of sorbitol in 20 mM histidine/50 mM nicotinamide solutions. The stability tests were performed under continuously stirring. PDSP prepared in 8 different formulations were placed in a50 mL beaker after filtration, ad the solutions were then stirred at 1,150 RPM at room temperature. These solutions were investigated every half a hour for the first 9 hours, as well as at 12-, 18- and 24-hours after the start of stirring. The times for precipitation and turbidity appearance were recorded.

FIG. 4 shows times for suspension, precipitation and turbidity to show up under continuously stirring conditions in PDSP formulations prepared with different concentrations of sorbitol in 20 mM histidine/150 mM nicotinamide solutions. Curve 1: time for suspended matter to show up. Curve 2: Time for visible precipitation to show up. Curve 3: Time for turbid solution to show up.

### DETAILED DESCRIPTION

Embodiments of the invention relate to formulations of PEDF-derived short peptides (PDSPs) with enhanced stabilities. Various human PDSPs were found to be promising therapeutics for treating or preventing various diseases or disorders, including muscle regeneration or arteriogenesis, alopecia and/or hair depigmentation, osteoarthritis, skin aging, liver cirrhosis, or eye diseases or conditions. Examples of such PDSPs may include those shown in TABLE 1:

TABLE 1
Examples of PEDF derived short peptides (PDSPs)

| | | SEQ | Human PEDF |
|---|---|---|---|
| Name | Sequence | ID NO | residues |
| 39 mer | LSVATALSALSLGAEQRTESIIHRALYYDLISSPDIHGT | 1 | 82-121 |
| 34 mer | ALSALSLGAEQRTESIIHRALYYDLISSPDIHGT | 2 | 88-121 |
| 29 mer | SLGAEQRTESIIHRALYYDLISSPDIHGT | 3 | 93-121 |
| 25 mer | EQRTESIIHRALYYDLISSPDIHGT | 4 | 97-121 |
| 24 mer | SLGAEQRTESIIHRALYYDLISSP | 5 | 93-116 |
| 20 mer | SLGAEQRTESIIHRALYYDL | 6 | 93-112 |
| 18 mer | EQRTESIIHRALYYDLIS | 7 | 97-114 |
| mo29 mer | SLGAEHRTESVIHRALYYDLITNPDIHST | 8 | mouse |
| mo20 mer | SLGAEHRTESVIHRALYYDL | 9 | mouse |

In accordance with embodiments of the invention, the PDSPs may be SEQ ID NO: 1, 2, 3, 5, 6, 8, or 9. In addition, the N-termini of these peptides may be optionally protected with acylation (e.g., acetyl or propionyl protection), and the C-termini may be optionally protected a amides.

These PDSPs have been prepared in citrate buffers and found to be effective for therapeutic purposes in various preclinical studies. However, preparation of these short peptides (e.g., PDSP (SEQ ID NO:3) in 10 mM citrate buffer with 0.85% w/v NaCl, pH 6.0) were found to lack long-term stabilities (over several months).

Many factors, including chemical stress (e.g., oxidation, hydrolysis, etc.) and physical stress (e.g., temperature, light, and agitation), can affect the qualities and stabilities of biopharmaceutical products, particularly during long-term storage. To investigate the stabilities of PDSP in different formulations, accelerated stability testing was performed. Specifically, various formulations were tested under stress conditions, particularly under shear stress, to identify optimal formulations. After extensive tests, certain formulations are unexpectedly found to have long-term stabilities superior to those of the original citrate buffer formulations.

The following describes specific examples to illustrate embodiments of the invention. However, one skilled in the art would appreciate that these specific examples are for illustration only and that other modifications and variations are possible without departing from the scope of the invention. For example, even though the following examples use PDSP (SEQ ID NO:3) for illustrations, other PDSPs may be used instead.

### 1. Citrate Buffer (10 mM Working Citrate Buffer with 0.85% w/v NaCl, pH6.0)

Citrate buffers were prepared from citrate acid and trisodium citrate to achieve the desired buffer capacity and pH. For example, citrate acid monohydrate (MW 210.14 kDa) (Merck) and trisodium citrate dihydrate (MW 294.12 kDa) (BioShop) were used to prepare solution A and solution B, respectively. These two solutions are then used to make the citrate buffers with the desired concentrations and pH values. The formula of solution A and B are as follows:

Solution A (0.1 M citrate acid monohydrate)(10 mli): 210.14 kDa×10/1000×0.1-0.21 g citrate acid monohydrate. Weigh 0.21 g Citrate acid monohydrate, and dissolve it in 10 mli ddH₂O to produce a 10 ml solution A stock.

Solution B (0.1 M trisodium citrate dihydrate)(10 ml): 294.12 kDa×10/1000×0.1-0.294 g trisodium citrate dihydrate. Wight 0.294 g Citrate acid monohydrate, and dissolve it in 10 ml ddH₂O to produce a 10 mi solution B stock.

To prepare a 10× citrate buffer stock pH 6.0, 1.15 ml solution A and 8.85 mi solution B were mixed to obtain a 0.1 M citrate buffer, 10 mL. Ten, the 10 ml, 0.1 M citrate buffer stock was diluted with 90 ml ddH₂O to generate a 10 mM working citrate buffer, 100 ml (1× solution).

To prepare a 10 mM citrate buffer with 0.35% w/v NaCl, 0.85 g NaCl was added into the 10 mM working citrate buffer, 100 ml. Before use, pH should be measured and adjusted based on study design.

### 2. Histidine Buffer (20 mM Histidine Buffer with 0-260 mM Sorbitol and/or 150-350 mM Nicotinamide, pH 7.0)

To prepare a 20 mL 20 mM Histidine buffer pH 7.0 for testing, 0.062 g histidine and different weights of sorbitol and/or nicotinamide were dissolved in 15 mL ddH₂O. Examples of various preparations with different sorbitol and nicotinamide concentrations ae prepared with the following compositions shown in TABLE 2:

TABLE 2
Various histidine buffer compositions

| | |
|---|---|
| 20 mM Histidine/150 mM Nicotinamide: | 0.37 g/20 ml |
| 20 mM Histidine/300 mM Nicotinamide: | 0.73 g/20 ml |
| 20 mM Histidine/350 mM Nicotinamide: | 0.86 g/20 ml |
| 20 mM Histidine/120 mM sorbitol: | 0.44 g/20 ml |
| 20 mM Histidine/140 mM sorbitol: | 0.51 g/20 ml |
| 20 mM Histidine/160 mM sorbitol: | 0.58 g/20 ml |
| 20 mM Histidine/180 mM sorbitol: | 0.66 g/20 ml |

The pH values of the buffers were adjusted to pH 7.0 using 2N NaOH or 1N HCl. The volumes of 2N NaOH or 1N HCl for pH value adjustment were recorded, and then ddH₂O was added to make a total volume of 20 ml.

### 3. Preparation of PDSP in Different Formulations

The PDSP used in these examples is a short synthetic peptide (29-mer) with acetylation at the NH2 terminus and amide at the COOH terminus. The molecular weight of PDSP is 3243.6 kDa. PDSP was dissolved in each of the solutions described above with the specific concentrations.

For example, to prepare a20 ml PDSP solution in a histidine/nicotinamide or a citrate buffer, 6772 mg of the peptide product was added to 20 mil histidine/nicotinamide buffer or citrate buffer.

The pH values of PDSP solutions were measured after PDSP completely dissolved in the solutions, and then the pH values were adjusted to 7.0 or 6.0 according to the study designs. Before use, PDSP solutions were each filtered trough a 0.2 μm syringe filter.

### 4. Stability Evaluation of PDSP in Different Formulation

We noticed that earlier formulations of PDSP in citrate buffers were not stable during long-term storage (over several months). To test the effects of different formulations on the stability, the various PDSP formulations were subject to stress conditions (e.g., shear stress) to accelerate the changes.

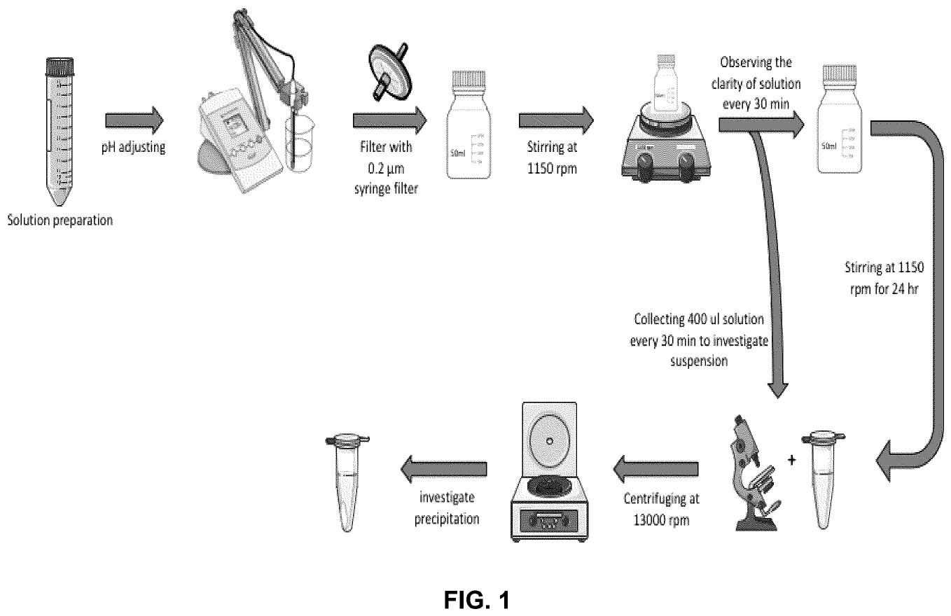

For these tests, twenty (20) milliliters of PDSP prepared in different buffers and excipients (a shown in Table 3) were each placed in a50 mL beaker after filtration. Then, the solutions were subject to stirring at 1,150 RPM at room temperature. Aliquots of 400 μl PDSP solutions each were collected into 1.5 ml Eppendorf tubes every half a hour up to 7 or 9 hours. The collected samples were centrifuged at 13,000 rpm for 5 min to evaluate whether ay precipitation had occurred. After the continuous observation, the stirring of PDSP solution was continued until 24 hours. The solution appearance and possible precipitation were investigated after 10-, 12-, 18- and 24-how stirring. The times for the appearance of suspended matter, precipitation, and turbidity were recorded. The experimental procedures are illustrated in FIG. 1.

TABLE 3
List of the formulations tested in this study.

| | | pH |
|---|---|---|
| Excipients | Base buffer | value |
| 0.85% NaCl | 10 mM citrate buffer | 6.0 |
| 150 mM Nicotinamide | 20 mM Histidine buffer | 7.0 |
| 300 mM Nicotinamide | 20 mM Histidine buffer | 7.0 |
| 350 mM Nicotinamide | 20 mM Histidine buffer | 7.0 |
| 260 mM Sorbitol | 20 mM Histidine buffer | 7.0 |
| 150 mM Nicotinamide, | 20 mM Histidine buffer | 7.0 |
| 120 mM Sorbitol | | |
| 150 mM Nicotinamide, | 20 mM Histidine buffer | 7.0 |
| 125 mM Sorbitol | | |
| 150 mM Nicotinamide, | 20 mM Histidine buffer | 7.0 |
| 130 mM Sorbitol | | |
| 150 mM Nicotinamide, | 20 mM Histidine buffer | 7.0 |
| 140 mM Sorbitol | | |
| 150 mM Nicotinamide, | 20 mM Histidine buffer | 7.0 |
| 150 mM Sorbitol | | |
| 150 mM Nicotinamide, | 20 mM Histidine buffer | 7.0 |
| 160 mM Sorbitol | | |
| 150 mM Nicotinamide, | 20 mM Histidine buffer | 7.0 |
| 170 mM Sorbitol | | |
| 150 mM Nicotinamide, | 20 mM Histidine buffer | 7.0 |
| 180 mM Sorbitol | | |

### RESULTS

### 1. The Ability to Results Shearing Force for PEDF-Derived Short Peptide (PDSP) Prepared in 10 mM Citrate Buffer with 0.85% w/v NaCl, pH 60

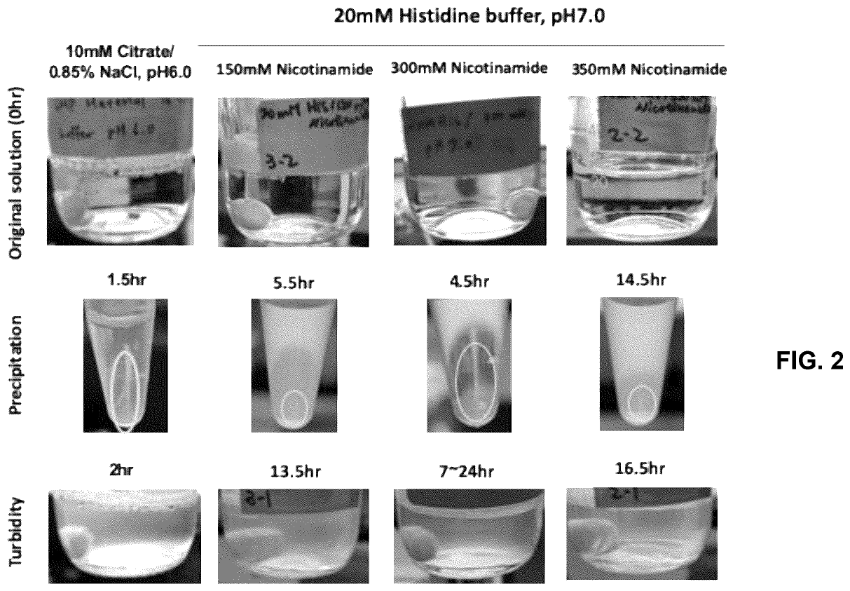

The original formulation for PDSP preparations 10 mM citrate buffer with 0.85% w/v NaCl, pH 6.0. This formulation was fine for various pre-clinical studies. However, thus formulation developed turbidity over a long-term storage (many months). Therefore, its stability was investigated using forced aggregation method to elucidate its ability to resist shearing force. As shown in FIG. 2, solution was clear and transparent before stirring (FIG. 2, upper panel). The suspended matter was seen in this formulation around 1 hour after the start of stirring (FIG. 2, left panel and Table 4). The precipitation and turbid solution were observed 1.5 mad 2.5 hours after start of the stirring, respectively. These observations will be used as baseline for comparison with other formulations.

TABLE 4
The stabilities of PDSP prepared in different formulations under stirring conditions

| Solution | | Suspension | Precipitation | Turbidity |
|---|---|---|---|---|
| Excipients/pH | Base buffer | (hour) | (hour) | (hour) |
| 0.85% NaCl, | 10 mM citrate | 1 | 1.5 | 2 |
| pH 6.0 | buffer | | | |
| 150 mM Nicotinamide, | 20 mM Histidine | 5 | 5.5 | 13.5 |
| pH 7.0 | buffer | | | |
| 300 mM Nicotinamide, | 20 mM Histidine | 4.5 | 4.5 | Between 7-24 |
| pH 7.0 | buffer | | | |
| 350 mM Nicotinamide, | 20 mM Histidine | 14 | 14.5 | 16.5 |
| pH 7.0 | buffer | | | |
| 260 mM Sorbitol, | 20 mM Histidine | 3 | 3 | 12 |
| pH 7.0 | buffer | | | |
| 150 mM Nicotinamide, | 20 mM Histidine | 3 | 4 | Between 9-12 |
| 120 mM Sorbitol, | buffer | | | |
| pH 7.0 | | | | |
| 150 mM Nicotinamide, | 20 mM Histidine | 4.5 | 6.5 | Between 9-24 |
| 125 mM Sorbitol, | buffer | | | |
| pH 7.0 | | | | |
| 150 mM Nicotinamide, | 20 mM Histidine | 4 | 5 | Between 9-12 |
| 130 mM Sorbitol, | buffer | | | |
| pH 7.0 | | | | |
| 150 mM Nicotinamide, | 20 mM Histidine | 13 | 13 | 18 |
| 140 mM Sorbitol, | buffer | | | |
| pH 7.0 | | | | |
| 150 mM Nicotinamide, | 20 mM Histidine | 13.5 | 14 | 18 |
| 150 mM Sorbitol, | buffer | | | |
| pH 7.0 | | | | |
| 150 mM Nicotinamide, | 20 mM Histidine | 6 | 6 | Between 9-12 |
| 160 mM Sorbitol, | buffer | | | |
| pH 7.0 | | | | |
| 150 mM Nicotinamide, | 20 mM Histidine | 5 | 5 | Between 9-12 |
| 170 mM Sorbitol, | buffer | | | |
| pH 7.0 | | | | |
| 150 mM Nicotinamide, | 20 mM Histidine | 4.5 | 4.5 | Between 9-12 |
| 180 mM Sorbitol, | buffer | | | |
| pH 7.0 | | | | |

### 2. The Abilities to Resist Shearing Forces for PEDF-Derived Short Peptides (PDSPs) Prepared in Histidine Buffers with Nicotinamide

To investigate the effect of nicotinamide, which is antioxidant, on PDSP stability in histidine-based buffer, PDSP prepared in 20 mM histidine buffer with 150 mM, 300 mM or 350 mM nicotinamide, pH 7.0, were chosen for comparison. As shown in FIG. 2 and Table 4, the suspended matters were observed at 5, 4.5 and 14 hours after the start or stirring for PDSP prepared in 20 mM histidine buffer with 150-, 300- and 350-mM nicotinamide, respectively (Table 4). The suspended matters in the formulations with histidine/nicotinamide buffers developed significantly later, a compared with formulations in citrate buffer.

In a study, the suspended matter in PDSP prepared in 10 mM citrate buffer with 0.85% NaCl was found to be coarse, and smell particles or fibers could be observed under dissection microscope. However, the suspended matter in the PDSP formulations prepared in histidine/nicotinamide buffer was very fine, which only decreased the solution transparency without visible particles under dissection microscope.

To evaluate whether precipitations could also occur with the suspended matter present, aliquots of 400 μl of each PDSP solutions were collected into a 1.5 ml Eppendorf tube for centrifugation (1,3000 rpm for 5 min). As shown in FIG. 2 (middle panel) and Table 4, the visible precipitation was observed at 5.5, 4.5 and 14.5 hours after the start of stirring for PDSP prepared in 20 mM histidine buffer with 150-, 300-, and 350-mM nicotinamide, respectively.

After suspended matter or precipitation was observed, the stirring of the PDSP solutions were continued until it turned turbid. FIG. 2 (lower panel) and Table 4 show that PDSP formulations prepared in 20 mM histidine buffer with 150-, 300-, and 350-mM nicotinamide became turbid at 13.5, 7-24, and 16.5 hours after the start of stirring, respectively.

Compared with PDSP formulations prepared in citrate buffers, PDSP formulations prepared in histidine/nicotinamide buffers can better withstand shearing stress. In addition, among these histidine/nicotinamide formulations, the solution with 350 mM nicotinamide showed longer time for precipitation to show up then the solution with 150 mM and 300 mM nicotinamide, suggesting that the higher concentration of nicotinamide could increase PDSP stability in formulations prepared in histidine-based buffers.

### 3. The Ability to Resist Shearing Force for PEDF-Derived Short Peptide (PDSP) Prepared in Histidine/Nicotinamide Buffer with Different Concentrations of Sorbitol

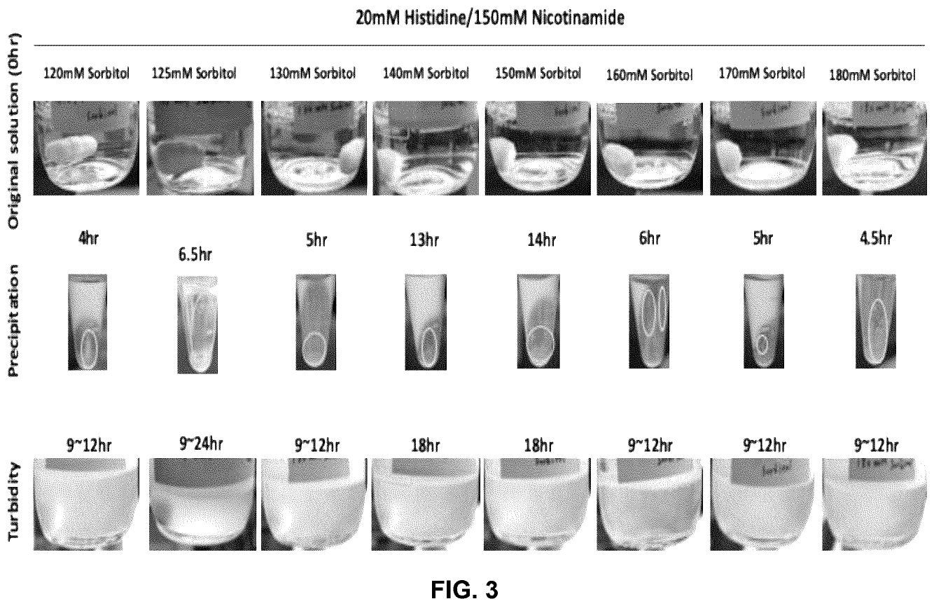

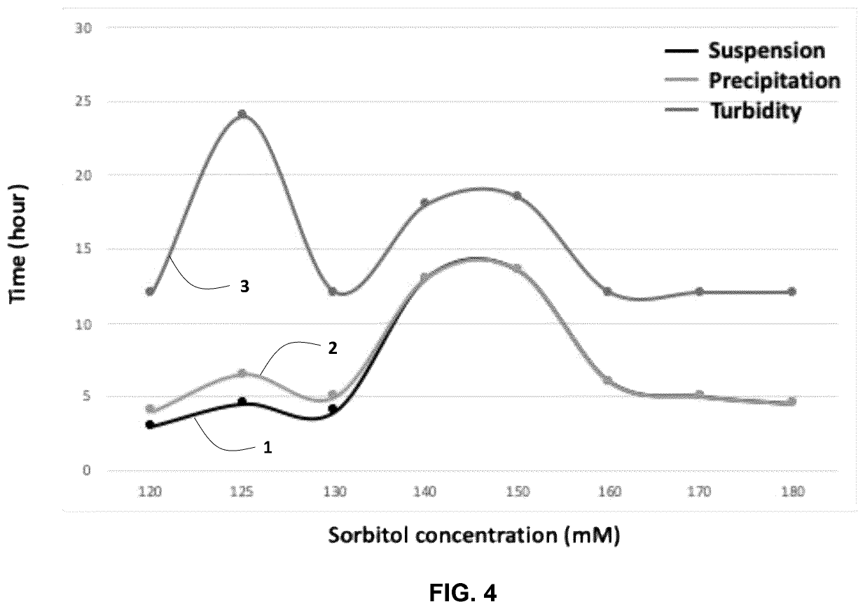

It has been reported that the ocular application of nicotinamide may cause eye irritation (Kari, G. 2005. Reassessment of the one experiment from the requirement of the tolerance for nicotinamide. United State Environmental Protection Agency Washington, D.C. 20460. 1-12). Thus, sorbitol was used to replace all or a portion of nicotinamide in histidine-based buffer. As shown in Table 4, the suspended matter was observed after stirring for just 3 hours in 20 mM histidine/260 mM sorbitol-only formulation. And the suspended matters were found at 3, 4.5, 4, 13, 13.5, 6, 5 and 4.5 hours, respectively, after the start of stirring in PDSP prepared in 20 mM Histidine/150 mM Nicotinamide buffers with 120-, 125-, 130-, 140-, 150-, 160-, 170- and 180-mM sorbitol (FIGS. 3, 4 and Table 4).

Among these Histidine/Nicotinamide formulations, the suspended mater, precipitation, and turbidity were observed after 13-hour continuously stirring in PDSP prepared in 20 mM Histidine/150 mM Nicotinamide with 140- and 150-mM Sorbitol, suggesting that the range from around 140 mM to 150 mM could be the optimal concentration for sorbitol in Histidine/Nicotinamide formulation. (FIG. 3, 4 and Table 4). Furthermore, the stabilities of PDSP prepared in 20 mM histidine/150 mM nicotinamide/140 or 150 mM sorbitol buffer and in 20 mM histidine/350 mM nicotinamide-only buffer were comparable, suggesting that sorbitol can be used to replace a portion of nicotinamide.

These results, together with data described above, suggest the formulations containing histidine/nicotinamide are better for PDSP stabilities the the citrate/NaCl formulations. Precipitations appeared at 1 hour after the start of stirring for PDSP prepared in 10 mM citrate with 0.85% NaCl, while the precipitation was observed after 5-how stirring for PDSP prepared in 20 mM histidine with 150 mM-350 mM nicotinamide. The 5 times longer duration for the histidine/nicotinamide formulations to develop precipitations indicate that the PDSPs are dramatically more stable in the histidine/nicotinamide formulations, as compared with the citrate/NaCl formulations. Furthermore, among the formations with different concentrations of nicotinamide, the precipitation was not observed until 14.5 hours after continuously stirring, indicating that the higher concentrations of nicotinamide are more suitable for maintaining PDSP stabilities an excipient.

Compared with PDSP formulations prepared in citrate buffers, PDSP formulations prepared in histidine/sorbitol-only buffer shows better ability to maintain PDSP stability. However, when compared with histidine/nicotinamide-only formulation, the capacity for maintaining PDSP stabilities we still not good enough for histidine/sorbitol-only formulations, indicating that nicotinamide may be a important component for the maintenance of PDSP stability in histidine-bed buffer.

When the tonicity agent, such a sorbitol, was used to replace a portion of nicotinamide, the time for precipitation appearance was similar for PDSP prepared in 20 mM histidine/350 mM nicotinamide (14.5 hours) and PDSP prepared in 20 mM histidine/150 mM nicotinamide/140 or 150 mM sorbitol (13 and 14 hours, respectively). These data further confirm that a concentration around 140-150 mM is a better choice of sorbitol concentrations for PDSP formulations prepared in 20 mM histidine/150 mM nicotinamide buffers.

Altogether, from the formulations we tested indicated that histidine/nicotinamide is a much better base buffer for formulations containing a PDSP (such a PDSP; SEQ ID NO:3) than the citrate buffers. According to embodiments of the invention, the PDSP may be at my suitable concentrations (such as 0.01%-5% w/v, preferably 0.01%-1% w/v) and histidine buffers may be used at any suitable concentrations, such as 1 mM-100 mM, preferably 5 mM-60 mM, more preferably 10 mM-40 mM, most preferably 15 mM-30 mM. The pH values for the formulations may be in a range from S to 9, preferably the pH values are around neutral, such a 6.5-7.5, most preferably around 7.0. The formulations comprise an antioxidant agent, preferably nicotinamide, at a suitable concentration, such as 50 mM-1000 mM, preferably 100 mM-700 mM, more preferably 200 mM-500 mM, ad most preferably 300 mM-400 mM. For example, a preferred formulation for PDSP solution may comprise 20 mM histidine with 350 mM nicotinamide, pH7.0. The formulations may also comprise a non-ionic tonicity agent, preferably sorbitol, at a suitable concentration, such as 0 mM-500 mM, preferably 10 mM-400 mM, more preferably 50 mM-300 mM, and most preferably 100 mM-200 mM. For example, a preferred formulation for PDSP solution may comprise 20 mM histidine with 150 mM nicotinamide and 150 mM sorbitol, pH7.0.

Formulations of the invention may be used to treat various diseases ad conditions, such as retinal degeneration, Meibomian glad disease, dry eye, etc. For eye applications, the formulations may be ophthalmic solutions.

Embodiment, of the invention have been illustrated with a limited number of examples. One skilled in the art would appreciate that these example are for illustration only and are not meant to limit the scope of the invention because other modifications and variations are possible without departing from the scope of the invention. Accordingly, the scope of the invention should be limited by the attached claim.

1. An aqueous formulation, comprising:
a PEDF-derived short peptide (PDSP) having the sequence of SEQ ID NO. 1, 2, 3, 5, 6, 8, or 9;
an antioxidant; and
histidine having a concentration of 1 mM-100 mM.

2. The aqueous formulation of claim 1, wherein a pH value is around 5-9.

3. The aqueous formulation of claim 1, wherein the antioxidant is nicotinamide.

4. The aqueous formulation of claim 3, wherein a concentration of nicotinamide is 50 mM-1000 mM.

5. The aqueous formulation of claim 1, further comprising a non-ionic tonicity agent.

6. The aqueous formulation of claim 5, wherein the non-ionic tonicity agent is sorbitol.

7. The aqueous formulation of claim 6, wherein a concentration of sorbitol is 0 mM-500 mM.

8. The aqueous formulation of claim 1, wherein the concentration of histidine is 5 mM-60 mM.

9. The aqueous formulation of claim 1, wherein the concentration of histidine is 10 mM-40 mM.

10. The aqueous formulation of claim 1, wherein the PDSP has the sequence of SEQ ID NO:3.

11. The aqueous formulation of claim 10, wherein a concentration of the PDSP is 0.01%-1% w/v.

12. The aqueous formulation of claim 2, wherein the PDSP has the sequence of SEQ ID NO:3.

13. The aqueous formulation of claim 3, wherein the PDSP has the sequence of SEQ ID NO:3.

14. The aqueous formulation of claim 4, wherein the PDSP has the sequence of SEQ ID NO:3.

15. The aqueous formulation of claim 5, wherein the PDSP has the sequence of SEQ ID NO:3.

16. The aqueous formulation of claim 6, wherein the PDSP has the sequence of SEQ ID NO:3.

17. The aqueous formulation of claim 7, wherein the PDSP has the sequence of SEQ ID NO:3.

18. The aqueous formulation of claim 8, wherein the PDSP has the sequence of SEQ ID NO:3.

19. The aqueous formulation of claim 9, wherein the PDSP has the sequence of SEQ ID NO:3.


SEQUENCE LISTING (9 sequences)

SEQ ID NO: 1 - protein, 39 aa, Artificial Sequence
  Synthetic
LSVATALSALSLGAEQRTESIIHRALYYDLISSPDIHGT

SEQ ID NO: 2 - protein, 34 aa, Artificial Sequence
  Synthetic
ALSALSLGAEQRTESIIHRALYYDLISSPDIHGT

SEQ ID NO: 3 - protein, 29 aa, Artificial Sequence
  Synthetic
SLGAEQRTESIIHRALYYDLISSPDIHGT

SEQ ID NO: 4 - protein, 25 aa, Artificial Sequence
  Synthetic
EQRTESIIHRALYYDLISSPDIHGT

SEQ ID NO: 5 - protein, 24 aa, Artificial Sequence
  Synthetic
SLGAEQRTESIIHRALYYDLISSP

SEQ ID NO: 6 - protein, 20 aa, Artificial Sequence
  Synthetic
SLGAEQRTESIIHRALYYDL

SEQ ID NO: 7 - protein, 18 aa, Artificial Sequence
  Synthetic
EQRTESIIHRALYYDLIS

SEQ ID NO: 8 - protein, 29 aa, Artificial Sequence
  Synthetic
SLGAEHRTESVIHRALYYDLITNPDIHST

SEQ ID NO: 9 - protein, 20 aa, Artificial Sequence
  Synthetic
SLGAEHRTESVIHRALYYDL